(19) United States
(12) Patent Application Publication
(10) Pub. No.: US 2021/0101604 A1
(43) Pub. Date: Apr. 8, 2021

(54) DRIVER STATE DETECTION DEVICE

(71) Applicant: KYUSHU INSTITUTE OF TECHNOLOGY, Kitakyushu-shi, Fukuoka (JP)

(72) Inventor: Yasushi SATO, Kitakyushu-shi (JP)

(73) Assignee: KYUSHU INSTITUTE OF TECHNOLOGY, Kitakyushu-shi, Fukuoka (JP)

(21) Appl. No.: 16/497,940

(22) PCT Filed: Mar. 8, 2018
(86) PCT No.: PCT/JP2018/008944

§ 371 Date: Sep. 26, 2019

(30) Foreign Application Priority Data
Mar. 28, 2017 (JP) 2017-063540

(51) Int. Cl. B60W 40/08; B60W 40/107; B60W 50/00; B60R 21/015

(52) U.S. Cl. CPC B60W 40/08; B60W 40/107; B60W 50/00; B60R 21/01526; B60W 2040/0827; B60W 2050/0008; B60W 2420/10; B60W 2420/60; B60W 2540/229; B60W 2540/221; B60W 2520/105; B60W 2040/0872

(57) ABSTRACT

A driver state detection device that accurately detects a state of a driver, such as fatigue and nodding off. A driver state detection device includes an acceleration sensor installed in a vehicle, a center-of-gravity movement amount detection unit that is attached to a component constituting the vehicle and detects a center-of-gravity movement amount of a body of a driver on the vehicle, and a driver state determination unit that determines a state of the driver based on magnitude of an amount of deviation between an acceleration of the vehicle obtained by the acceleration sensor and the center-of-gravity movement amount of the body of the driver detected by the center-of-gravity movement amount detection unit.

### TECHNICAL FIELD

The present invention relates to a driver state detection device.

### BACKGROUND ART

There is a conventionally known method of extracting fluctuation components contained in pulse waves by analyzing the frequency of pulse wave data at RR intervals defining the heart rate, and analyzing the extracted fluctuation components, to detect the subject's fatigue or nodding off (sleep onset timing), for example. For example, Patent Literature 1 discloses a technique of detecting the subject's sleep onset timing based on information on the presence or absence of body movement of the subject, and on information on the ratio of sympathetic nerve components and the ratio of parasympathetic nerve components to the fluctuation components of the subject's heart rate. Such a technique of detecting fatigue, nodding off, or the like of a subject is expected to be utilized also in the technical field of safe driving support for a driver who drives a vehicle such as a car or a train.

However, under conditions where vibrations are always generated in the human body, such as during driving of a vehicle, the waveform of the pulse waves tends to collapse, and therefore, it is difficult to detect fluctuation components from pulse wave data. That is, it is not an easy task to accurately detect the degree of fatigue, nodding off, or other state of a driver who drives a vehicle with the conventional method of detecting the state of the subject by analyzing the frequency of pulse wave data using the technique described in Patent Literature 1, for example.

As a technique for solving this problem, for example, Patent Literature 2 discloses a technique of detecting the degree of arousal or fatigue of a driver based on information on changes over time in the body pressure distribution of the driver obtained from output signals of pressure sensors disposed on a seat on which the driver is seated.

### CITATION LIST

### Patent Literature

Patent Literature 1: JP 2012-020117 A
Patent Literature 2: JP 10-129016 A

### SUMMARY OF INVENTION

### Technical Problem

The technique described in Patent Literature 2 determines the center of gravity of the body pressure distribution of the driver and detects the body movement of the driver from the movement amount of the center of gravity. When no change in body movement is detected for a first predetermined period of time or longer after a detection of body movement, it is determined that the driver's fatigue is accumulated. In addition, when body movement is detected a predetermined number of times within a second predetermined period of time shorter than the first predetermined period of time, it is determined that the driver's degree of arousal is low. However, in the technique described in Patent Literature 2, for example, even if the driver is driving for a long time while maintaining the same posture in a state with a high degree of arousal, an erroneous determination on drowsy driving might be made. Therefore, in the technical field of safe driving support for drivers, there has been a need to provide a technique of accurately detecting a state of a driver, such as fatigue and nodding off.

The present invention has been made in view of the above-described situation, and an object of the present invention is to provide a driver state detection device that accurately detects a state of a driver, such as fatigue and nodding off.

### Solution to Problem

In order to solve the above-described problem, a driver state detection device according to the present invention includes an acceleration sensor installed in a vehicle, a center-of-gravity movement amount detection unit, and a driver state determination unit. The center-of-gravity movement amount detection unit is attached to a component that constitutes the vehicle and detects the center-of-gravity movement amount of the body of a driver on the vehicle. The driver state determination unit determines the state of the driver based on the magnitude of the amount of deviation between the acceleration of the vehicle obtained by the acceleration sensor and the center-of-gravity movement amount of the body of the driver detected by the center-of-gravity movement amount detection unit.

### Advantageous Effects of Invention

The driver state detection device according to the present invention can accurately detect the state of the driver, such as fatigue and nodding off.

### BRIEF DESCRIPTION OF DRAWINGS

FIG. 1 is a block diagram illustrating a schematic configuration of a driver state detection device according to an embodiment of the present invention.

FIG. 2 is a top view of the inside of a vehicle illustrating an implementation example of an acceleration sensor and a posture sensor according to an embodiment of the present invention on a vehicle.

FIG. 3 is a schematic view illustrating a configuration example of a posture sensor according to an embodiment of the present invention.

FIG. 4 is a block diagram illustrating a configuration example of an X-direction error signal generation unit according to an embodiment of the present invention.

FIG. 5 is a block diagram illustrating a configuration example of a driver state determination unit according to an embodiment of the present invention.

FIG. 6 is an explanatory view illustrating a state in which a Doppler sensor according to a modification is attached to a seat belt worn by a driver.

FIG. 7 is an explanatory view illustrating a state in which a polymer thick film sensor or a minute vibration detection microphone according to a modification is attached to the seat belt worn by the driver.

### DESCRIPTION OF EMBODIMENTS

Hereinafter, details of the driver state detection device according to an embodiment of the present invention will be described with reference to the drawings.

### [Schematic Configuration of Driver State Detection Device]

First, with reference to FIG. 1, a schematic configuration of a driver state detection device according to the present embodiment will be described. FIG. 1 is a block diagram illustrating a schematic configuration of a driver state detection device 100 according to the present embodiment. As illustrated in FIG. 1, the driver state detection device 100 includes an acceleration sensor 1, a posture sensor 2 (an example of a center-of-gravity movement amount detection unit), a center-of-gravity movement amount calculation unit 3 (an example of a center-of-gravity movement amount detection unit), an error signal generation unit 4, and a driver state determination unit 5 (an example of a driver state determination unit).

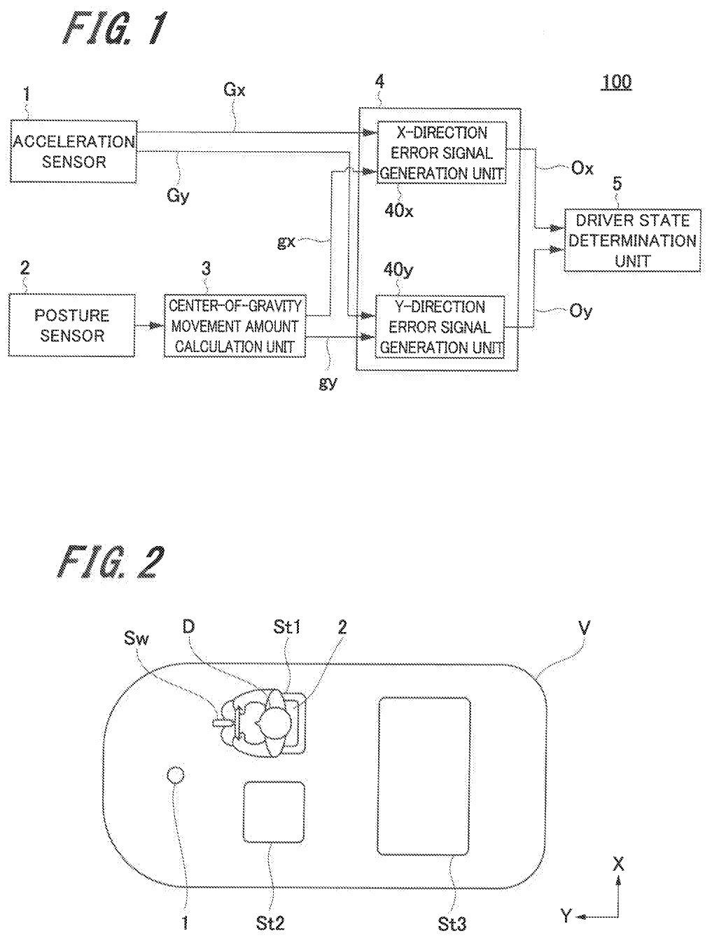

The acceleration sensor 1 is attached to a vehicle V (see FIG. 2), and outputs accelerations Gx and Gy of the vehicle V obtained by measurement to the error signal generation unit 4. The acceleration Gx is an acceleration applied in the X direction which is the vehicle width direction of the vehicle V, and the acceleration Gy is an acceleration applied in the Y direction which is the longitudinal direction of the vehicle V. The acceleration sensor 1 can be composed of, for example, a six-axis acceleration sensor, and may be alternatively composed of a three-axis acceleration sensor. In the following description, the acceleration Gx in the X direction and the acceleration Gy in the Y direction are simply referred to as the acceleration G when it is not necessary to distinguish them individually.

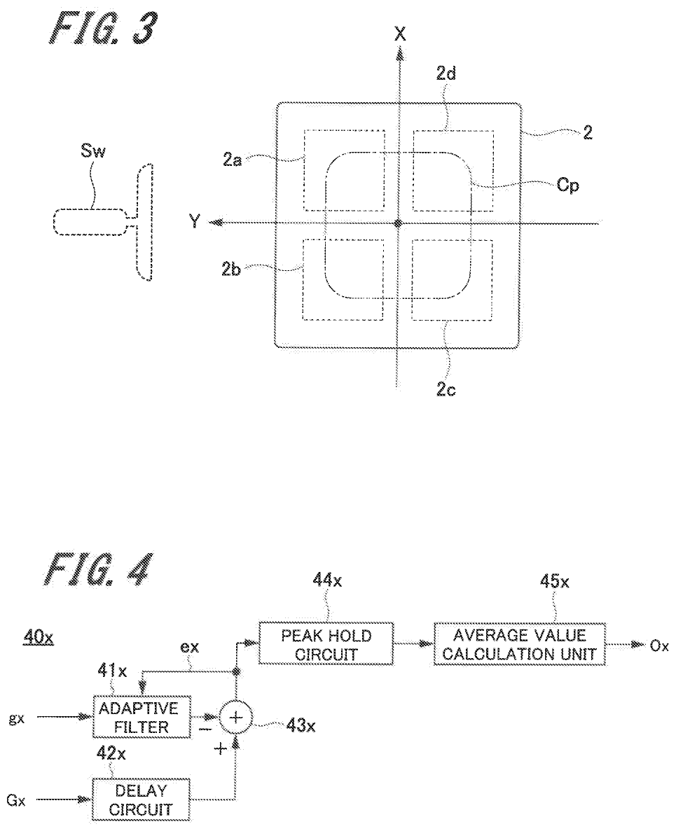

The posture sensor 2 is a sensor disposed on the upper surface of a seat St1 (an example of a driver's seat, see FIG. 2) on which a driver driving the vehicle V (an example of a driver) is seated, and includes an incorporated film-type piezoelectric sensor (see FIG. 3). The posture sensor 2 generates an output signal according to the strength of the pressure applied by the driver sitting on the seat St1, and outputs the output signal to the center-of-gravity movement amount calculation unit 3. The posture sensor 2 will be described in detail with reference to FIG. 3 described later.

The center-of-gravity movement amount calculation unit 3 calculates the amount of movement of the body (hereinafter referred to as the center-of-gravity movement amount) generated with the movement of the driver's center of gravity based on the output signal from the posture sensor 2. The center-of-gravity movement amount calculation unit 3 calculates a center-of-gravity movement amount gx in the X direction and a center-of-gravity movement amount gy in the Y direction as the center-of-gravity movement amount. The center-of-gravity movement amount calculation unit 3 will be described in detail with reference to FIG. 3 illustrating an example of the configuration of the posture sensor 2. In the following description, when it is not necessary to distinguish the center-of-gravity movement amount gx in the X direction and the center-of-gravity movement amount gy in the Y direction, they are simply referred to as a center-of-gravity movement amount g.

The error signal generation unit 4 includes an X-direction error signal generation unit 40x and a Y-direction error signal generation unit 40y. The X-direction error signal generation unit 40x generates an X-direction error signal Ox based on the acceleration Gx input from the acceleration sensor 1 and the center-of-gravity movement amount gx of the driver input from the center-of-gravity movement amount calculation unit 3. The Y-direction error signal generation unit 40y generates a Y-direction error signal Oy based on the acceleration Gy input from the acceleration sensor 1 and the center-of-gravity movement amount gy of the driver input from the center-of-gravity movement amount calculation unit 3. The error signal generation unit 4 will be described in detail with reference to FIG. 4 described later.

The driver state determination unit 5 adds the X-direction error signal Ox and the Y-direction error signal Oy to generate an error signal O, and determines the state of the driver based on a result obtained by comparing the generated error signal O with a preset threshold. The driver state determination unit 5 will be described in detail with reference to FIG. 5 described later.

### [Implementation Example of Acceleration Sensor and Posture Sensor]

Next, with reference to FIG. 2, an implementation example of the acceleration sensor 1 and the posture sensor 2 on the vehicle V will be described. FIG. 2 is a top view of the inside of the vehicle illustrating an implementation example of the acceleration sensor 1 and the posture sensor 2 on the vehicle V. As illustrated in FIG. 2, the vehicle V is provided with a seat St1 on which a driver D operating a steering wheel Sw is seated, a seat St2 serving as a passenger seat, and a seat St3 serving as a rear seat.

The acceleration sensor 1 for detecting the acceleration G of the vehicle V is installed at a position forward of the seat St1 and the seat St2 in the longitudinal direction (Y direction) of the vehicle V. The installation position of the acceleration sensor 1 illustrated in FIG. 2 is an example, and the acceleration sensor 1 may be installed at another position inside the vehicle V.

The posture sensor 2 is disposed on the seat surface of the seat St1 on which the driver D is seated. The posture sensor 2 is formed in a flat plate-like shape like a cushion, and on the upper surface thereof, the buttocks of the driver D seated on the seat St1 are disposed.

### [Configuration Example of Posture Sensor]

Next, with reference to FIG. 3, a configuration example of the posture sensor 2 will be described. FIG. 3 is a schematic view illustrating a configuration example of the posture sensor 2. The posture sensor 2 is composed of four film-type piezoelectric sensors 2a to 2d. The four piezoelectric sensors 2a to 2d are disposed in respective regions obtained by dividing the coordinate plane of the seat surface of the seat St1 (an example of a component constituting the vehicle) on which the driver D is seated into four.

The position of the center surrounded by the four piezoelectric sensors 2a to 2d is the origin in the coordinate plane, the vertical direction in the figure corresponds to the X axis (the vehicle width direction of the vehicle V), and the horizontal direction corresponds to the Y axis (the longitudinal direction of the vehicle V). The piezoelectric sensor 2a is disposed at a position corresponding to the first quadrant of the coordinate plane, the piezoelectric sensor 2b is disposed at a position corresponding to the second quadrant, the piezoelectric sensor 2c is disposed at a position corresponding to the third quadrant, and the piezoelectric sensor 2d is disposed at a position corresponding to the fourth quadrant. The positive direction along the X axis in the figure (the direction in which the piezoelectric sensors 2a and 2d are arranged) corresponds to the right direction for the driver D, and the negative direction (the direction in which the piezoelectric sensors 2b and 2c are arranged) corresponds to the left direction. Further, the positive direction along the Y axis in the figure (the direction in which the piezoelectric sensors 2a and 2b are arranged) corresponds to the forward direction for the driver D (the direction in which the steering wheel Sw is positioned), and the negative direction (the direction in which the piezoelectric sensors 2c and 2d are arranged) corresponds to the backward direction for the driver D.

The center-of-gravity movement amount calculation unit 3 calculates the center-of-gravity movement amount g using information on the arrangement position of the piezoelectric sensors 2a to 2d on the coordinate plane and values of output signals from the piezoelectric sensors 2a to 2d. Specifically, the center-of-gravity movement amount calculation unit 3 calculates, as the center-of-gravity movement amount gx of the driver D in the X direction, a difference between output signals from the piezoelectric sensors 2a and 2d arranged in the region on the positive side along the X axis and output signals from the piezoelectric sensors 2b and 2c arranged in the region on the negative side along the X axis. The center-of-gravity movement amount gx calculated in this manner is output from the center-of-gravity movement amount calculation unit 3 as a waveform indicating the magnitude of swing of the body in the lateral (right and left) direction of the driver D and the direction of the swing.

In addition, the center-of-gravity movement amount calculation unit 3 calculates, as the center-of-gravity movement amount gy of the driver D in the Y direction, a difference between output signals from the piezoelectric sensors 2a and 2b arranged in the region on the positive side along the Y axis and output signals from the piezoelectric sensors 2c and 2d arranged in the region on the negative side along the Y axis. The center-of-gravity movement amount gy calculated in this manner is output from the center-of-gravity movement amount calculation unit 3 as a waveform indicating the magnitude of swing of the body in the vertical (back and forth) direction of the driver D and the direction of the swing.

In the example illustrated in FIG. 3, the buttocks of the driver D seated on the seat St1 provided with the posture sensor 2 are at a position Cp near the center of the posture sensor 2. In this case, pressure from the body of the driver D is uniformly applied to each of the four piezoelectric sensors 2a to 2d constituting the posture sensor 2. In this state, if no acceleration G is applied to the vehicle V, the movement of the body of the driver D hardly occurs, so that the center-of-gravity movement amount gx and the center-of-gravity movement amount gy calculated by the center-of-gravity movement amount calculation unit 3 are values close to “0”.

On the other hand, in a state in which the acceleration Gx in the lateral (X) direction is applied to the vehicle V, for example, the body of the driver D swings rightward and leftward so as to follow the direction in which the acceleration Gx is applied. That is, the output value of the center-of-gravity movement amount gx is larger than the output value of the center-of-gravity movement amount gy. At this time, if the driver D is in a normal (awake) state in which the driver D is not nodding off, for example, the driver D unconsciously moves his or her body (holds on) in the direction opposite to the direction in which the acceleration Gx is applied, on the basis of the position control based on the human sense of balance. Therefore, for example, when the center-of-gravity movement amount gx and the acceleration Gx of the vehicle V are plotted on a graph in which the vertical axis represents the X direction of the vehicle V and the horizontal axis represents time, the waveform illustrating the center-of-gravity movement amount gx and the waveform illustrating the acceleration Gx of the vehicle V have a small difference in the vertical axis direction of the graph.

On the other hand, in a state in which the driver D is suffering from accumulated fatigue, in a state in which the driver is nodding off, for example, the above-described position control is not in effect. In the case where the acceleration Gx in the lateral direction is applied to the vehicle V, the body of the driver D swings largely in the direction in which the acceleration Gx is applied. Therefore, the waveform indicating the center-of-gravity movement amount gx is largely deviated from the waveform indicating the acceleration Gx of the vehicle V in the vertical axis direction of the graph. That is, it can be considered that the amount of deviation between the center-of-gravity movement amount g of the driver D and the acceleration Gx of the vehicle V represents the transfer characteristics of the position control performed based on the human sense of balance.

The driver state detection device 100 according to the present embodiment detects the state of the driver D, such as the degree of fatigue or nodding off, based on the magnitude of the amount of deviation between the center-of-gravity movement amount g of the driver D and the acceleration Gx of the vehicle V. Specifically, the error signal generation unit 4 generates an error signal according to the amount of deviation between the center-of-gravity movement amount g of the driver D and the acceleration Gx of the vehicle V, and the driver state determination unit 5 compares the value of the error signal with a threshold previously associated with the state of the driver D to determine the state of the driver D.

### [Configuration Example of Error Signal Generation Unit]

Next, a configuration example of the error signal generation unit 4 will be described. Although the error signal generation unit 4 includes the X-direction error signal generation unit 40x and the Y-direction error signal generation unit 40y, the both have the same configuration, and therefore, the X-direction error signal generation unit 40x will be described herein as an example. FIG. 4 is a block diagram illustrating a configuration example of the X-direction error signal generation unit 40x. As illustrated in FIG. 4, the X-direction error signal generation unit 40x includes an adaptive filter 41x, a delay circuit 42x, a subtractor 43x, a peak hold circuit 44x, and an average value calculation unit 45x.

Two input terminals of the adaptive filter 41x are respectively connected to an output terminal (not illustrated) of the acceleration sensor 1 (see FIG. 1) and an output terminal of the subtractor 43x. An output terminal of the adaptive filter 41x is connected to a “−” input terminal of the subtractor 43x. An input terminal of the delay circuit 42x is connected to the center-of-gravity movement amount calculation unit 3 (see FIG. 1), and an output terminal of the delay circuit 42x is connected to a “+” input terminal of the subtractor 43x. The output terminal of the subtractor 43x is connected to one input terminal of the adaptive filter 41x and an input terminal of the peak hold circuit 44x. An output terminal of the peak hold circuit 44x is connected to an input terminal of the average value calculation unit 45x.

The adaptive filter 41x is composed of, for example, an LMS (least mean square) filter. The adaptive filter 41x updates a filter coefficient to minimize the value of an error signal gx indicating the difference between the output from the adaptive filter itself (hereinafter referred to as “filter output”) and the acceleration Gx of the vehicle V input from the acceleration sensor 1. The adaptive filter 41x then performs a convolution operation between the updated filter coefficient and the center-of-gravity movement amount gx input from the center-of-gravity movement amount calculation unit 3, and outputs the calculation result as the filter output. The filter output from the adaptive filter 41x is input to the “−” input terminal of the subtractor 43x.

The delay circuit 42x adds, to the acceleration Gx input from the acceleration sensor 1, a delay corresponding to the time taken for operation performed by the adaptive filter 41x. The acceleration Gx to which the delay is added is input to the “+” input terminal of the subtractor 43x.

The subtractor 43x subtracts the filter output input from the adaptive filter 41x from the acceleration Gx input from the delay circuit 42x to generate an error signal ex. The error signal ex is a signal indicating the difference between the acceleration Gx input from the delay circuit 42x and the center-of-gravity movement amount gx on which the adaptive filter 41x has performed the convolution operation with the filter coefficient. Therefore, the value of the error signal ex becomes a larger value as the difference between the center-of-gravity movement amount gx and the acceleration Gx increases.

The error signal ex generated by the subtractor 43x is input to the input terminal of the adaptive filter 41x and the input terminal of the peak hold circuit 44x. The peak hold circuit 44x performs processing of holding a peak value of the error signal ex output from the subtractor 43x, and outputs the held peak value to the average value calculation unit 45x. The average value calculation unit 45x holds the peak value output from the peak hold circuit 44x for a predetermined period of time, such as one second, calculates the average value thereof, and outputs the calculated average value as the error signal Ox. That is, the peak hold circuit 44x and the average value calculation unit 45x in the present embodiment have a function as an LPF (Low Pass Filter) that removes noise components included in the error signal ex.

The value of the error signal Ox (or the error signal Oy) output from the error signal generation unit 4 becomes smaller in a state where the driver D is awake and the position control is in effect, and becomes larger in a state where the driver D is suffering from accumulated fatigue, or nodding off, for example.

In the present embodiment, an example in which the error signal Ox is generated from the error signal ex (the error signal Oy is generated from the error signal ey) output from the subtractor 43 has been described, but the present invention is not limited to this. The error signal Ox (or Oy) may be generated by inputting the filter output of the adaptive filter 41x (or 41y (not illustrated)) to the peak hold circuit 44x (or 44y (not illustrated)).

### [Configuration Example of Driver State Determination Unit]

Next, a configuration example of the driver state determination unit 5 will be described with reference to FIG. 5. FIG. 5 is a block diagram illustrating a configuration example of the driver state determination unit 5. As illustrated in FIG. 5, the driver state determination unit 5 includes an X-direction gain adjustment unit 51x, a Y-direction gain adjustment unit 51y, an adder 52, and a threshold comparison unit 53.

An input terminal of the X-direction gain adjustment unit 51x is connected to an output terminal of the average value calculation unit 45x of the X-direction error signal generation unit 40x (see FIG. 1), and an output terminal of the X-direction gain adjustment unit 51x is connected to one input terminal of the adder 52. An input terminal of the Y-direction gain adjustment unit 51y is connected to an output terminal of an average value calculation unit (not illustrated) of the Y-direction error signal generation unit 40y (see FIG. 1), and an output terminal of the Y-direction gain adjustment unit 51y is connected to the other input terminal of the adder 52. An output terminal of the adder 52 is connected to an input terminal of the threshold comparison unit 53.

The X-direction gain adjustment unit 51x adds a predetermined gain to the error signal Ox output from the average value calculation unit 45x of the X-direction error signal generation unit 40x, and outputs the result. The Y-direction gain adjustment unit 51y adds a predetermined gain to the error signal Oy output from (the average value calculation unit of) the Y-direction error signal generation unit 40y, and outputs the result. Each gain set in the X-direction gain adjustment unit 51x and the Y-direction gain adjustment unit 51y is set to any desired value selected by the user who uses the driver state detection device 100. The user can set more gains with respect to the gain adjustment unit 51 corresponding to the direction in which the user wishes to more intensively determine the information on the state of the driver D in the X direction or the Y direction.

The adder 52 adds the error signal Ox whose gain is adjusted by the X-direction gain adjustment unit 51x and the error signal Oy whose gain is adjusted by the Y-direction gain adjustment unit 51y, and outputs the error signal O obtained by the adding to the threshold comparison unit 53.

The threshold comparison unit 53 compares the error signal O output from the adder 52 with a threshold set in advance in association with the state of the driver D, and determines the state of the driver D based on the result of the comparison. For example, when the value of the error signal O exceeds a threshold that can lead to a determination that the driver D is suffering from accumulated fatigue, the threshold comparison unit 53 determines that the driver D is suffering from accumulated fatigue. When the value of the error signal O exceeds a threshold that can lead to a determination that the driver D is in the nodding-off state, the threshold comparison unit 53 determines that the driver D is in the nodding-off state. The thresholds set in the threshold comparison unit 53 can be set to optimal values obtained by an experiment or the like.

### [Various Effects]

In the above-described embodiment, the driver state determination unit 5 compares the magnitude of the amount of deviation between the acceleration G of the vehicle V obtained by the acceleration sensor 1 and the center-of-gravity movement amount g of the driver D detected by the posture sensor 2 and the center-of-gravity movement amount calculation unit 3 with thresholds, thereby determining the state of the driver D. It is considered that the error signal O indicating the amount of deviation between the acceleration G of the vehicle V and the movement amount g of the center of gravity of the body of the driver D is a value representing the transfer characteristics of the position control performed based on the human sense of balance as described above. That is, according to the above-described embodiment, the state of the driver D, such as fatigue and nodding off, can be accurately detected based on the magnitude of the value of the error signal O indicating the transfer characteristics of the human position control.

Further, according to the above-described embodiment, other states, such as a state of being sick, can be determined as long as these states of the driver D can be determined from the transfer characteristics of the position control. Since the effectiveness of the position control also varies depending on the driving skill of the driver D, according to the above-described embodiment, the driving skill (mastery) of the driver D can also be determined.

Further, in the above-described embodiment, the error signal generation unit 4 includes the adaptive filter 41. The adaptive filter 41 updates the filter coefficient to minimize the value of the error signal e indicating the difference between the filter output obtained by the convolution of the center-of-gravity movement amount g of the driver D and the filter coefficient and the acceleration G of the vehicle V. Then, based on the value of the error signal O, which is a signal obtained by removing noise components from the error signal e indicating the difference between the filter output of the adaptive filter 41 and the acceleration G of the vehicle V, the driver state determination unit 5 determines the state of the driver D. Thus, according to the above-described embodiment, the state of the driver D, such as fatigue and nodding off, can be detected accurately with a simple configuration.

In addition, even if the driver D is awake and the position control based on the sense of balance is in effect, a predetermined delay occurs until the position of the body of the driver D is actually controlled after the acceleration G is applied to the vehicle V (the brain detects the acceleration). In the above-described embodiment, since the adaptive filter 41 can absorb this delay, the state of the driver D can be detected accurately.

In the above-described embodiment, the center-of-gravity movement amount g of the driver D is calculated based on the output value of the posture sensor 2 including the piezoelectric sensors 2a to 2d provided on the seat surface of the seat St1, which is the driver's seat of the vehicle V. Then, the state of the driver D is determined based on the magnitude of the amount of deviation between the center-of-gravity movement amount g and the acceleration G of the vehicle V. Therefore, according to the above-described embodiment, it is possible to accurately detect the state of the driver D in a contactless manner without attaching any sensor or the like to the body of the driver D.

### [Various Modifications]

While in the above-described embodiment, the error signal generation unit 4 includes the X-direction error signal generation unit 40X and the Y-direction error signal generation unit 40Y, which separately generate the error signal Ox in the X direction and the error signal Oy in the Y direction, the present invention is not limited to this. For example, the center-of-gravity movement amount calculation unit 3 may be configured to add the center-of-gravity movement amount gx and the center-of-gravity movement amount gy to generate a center-of-gravity movement amount g′, and the error signal generation unit 4 may be configured to generate the error signal O based on the center-of-gravity movement amount g′. The center-of-gravity movement amount g′ can be calculated, for example, by the following Expression 1.

Center-of-gravity movement amount g′=A·Sin(θ) Expression 1

In Expression 1 above, “A” indicates “r” (moving radius) in polar coordinates, and “θ” indicates a declination. In Expression 1 above, “A” can be determined by the following Expression 2, and “θ” can be determined by the following Expression 3.

$$\begin{matrix}[ Formula 1 ] & \\ A = \sqrt{\begin{matrix}Center \text{-} of \text{-} gravity movement amount {gx}^{2} + \\ Center \text{-} of \text{-} gravity movement amount {gy}^{2}\end{matrix}} & Expression 2 \\ [ Formula 2 ] & \\ θ = {tan}^{- 1} \frac{Center \text{-} of \text{-} gravity movement amount gy}{Center \text{-} of \text{-} gravity movement amount gx} & Expression 3\end{matrix}$$

In a method in which the center-of-gravity movement amount calculation unit calculates the center-of-gravity movement amount g′, only one adaptive filter 41 needs to be provided corresponding to the center-of-gravity movement amount g′, and the amount of operation can be reduced compared with the above-described embodiment.

Further, the above-described embodiment provides an example in which, based on the information on the amount of deviation between the acceleration G of the vehicle V detected by the acceleration sensor 1 and the center-of-gravity movement amount g of the driver D detected by the posture sensor 2 and the center-of-gravity movement amount calculation unit 3, the driver state determination unit 5 detects the state of the driver D. However, the present invention is not limited to this example. The posture sensor for detecting the center-of-gravity movement amount g of the driver D to be compared with the acceleration G of the vehicle V detected by the acceleration sensor 1 may be composed of another sensor.

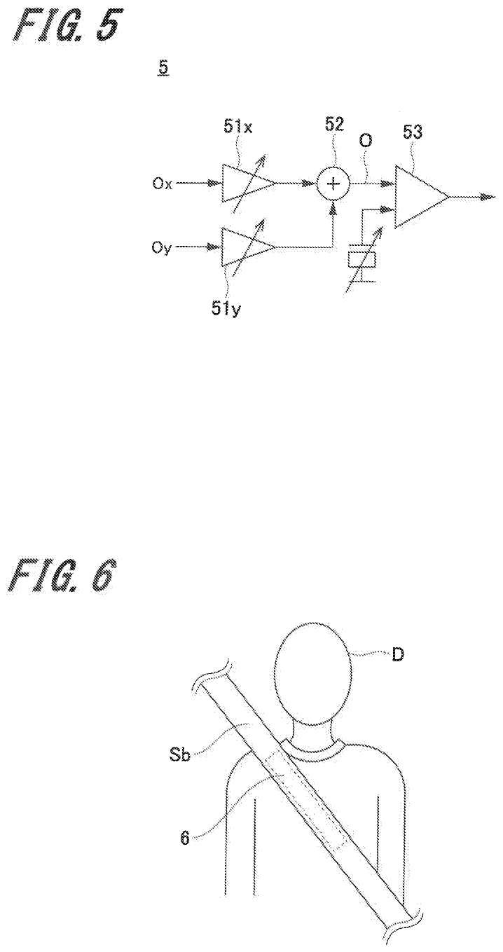

For example, the posture sensor may be composed of a radio wave sensor, such as a Doppler sensor. An example in which the posture sensor is composed of a Doppler sensor will be described with reference to FIG. 6. FIG. 6 is an explanatory view illustrating a state in which a Doppler sensor 6 is attached to a seat belt Sb worn by the driver D.

In the example illustrated in FIG. 6, the Doppler sensor 6 is attached at a position corresponding to the chest of the driver D in the seat belt Sb worn by the driver D. The Doppler sensor 6 emits radio waves (microwaves) toward the body of the driver D. By comparing (Doppler sensing) the frequency of the radio waves reflected on the body of the driver D back to the sensor with the frequency of the emitted radio waves, the Doppler sensor 6 detects the movement of the body of the driver D.

For example, when the distance between the Doppler sensor 6 and the body of the driver D decreases due to, for example, the body of the driver D tilting in the direction toward the steering wheel Sw (see FIGS. 2 and 3), the polarity of the movement speed of the body of the driver D detected by the Doppler sensor 6 becomes positive. Then, the frequency of the radio waves reflected on the body of the driver D back to the sensor, which are detected by the Doppler sensor 6, becomes high. On the other hand, when the distance between the Doppler sensor 6 and the body of the driver D increases due to the body of the driver D moving away from the steering wheel Sw, the polarity of the movement speed of the body of the driver D detected by the Doppler sensor 6 becomes negative. Then, the frequency of the radio waves reflected on the body of the driver D back to the sensor, which are detected by the Doppler sensor 6, becomes low.

That is, output values from the Doppler sensor 6 illustrated in FIG. 6 include information on the distance between the seat belt Sb on which the Doppler sensor 6 is attached and the body of the driver D, and the movement direction and the movement speed of the body of the driver D in the front-rear direction (Y direction) of the body.

The output values from the Doppler sensor 6 can be input to the Y-direction error signal generation unit 40y (see FIG. 1). Accordingly, the Y-direction error signal generation unit 40y compares the output values from the Doppler sensor 6 with the acceleration Gy in the Y direction of the vehicle V obtained by the acceleration sensor 1. Then, the error signal ey (not illustrated) indicating the magnitude of the error between the two values is output from the Y-direction error signal generation unit 40y to the driver state determination unit 5 (see FIG. 1). The driver state determination unit 5 determines the state of the driver D by comparing the error signal Oy obtained by removing noise components from the error signal ey with a threshold.

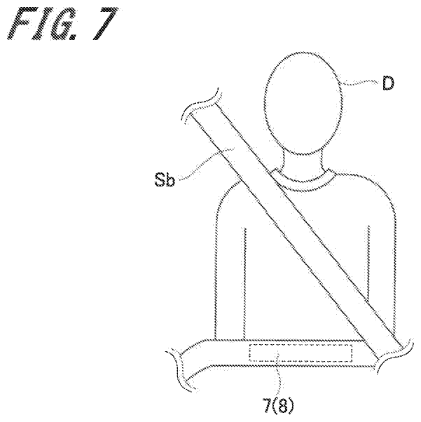

In addition, the posture sensor may be composed of a pressure sensor, such as a polymer thick film sensor, or a minute vibration detection microphone. An example in which the posture sensor is composed of a polymer thick film sensor or a minute vibration detection microphone will be described with reference to FIG. 7. FIG. 7 is an explanatory view illustrating a state in which a polymer thick film sensor 7 or a minute vibration detection microphone 8 is attached to the seat belt Sb worn by the driver D.

In the example illustrated in FIG. 7, the polymer thick film sensor 7 or the minute vibration detection microphone 8 is attached to a position corresponding to the abdomen of the driver D in the seat belt Sb worn by the driver D. First, an example in which the polymer thick film sensor 7 is attached to the seat belt Sb will be described. The polymer thick film sensor 7 is a sensor having a characteristic of decreasing electrical resistance value with an increase in pressure applied to the sensor. Therefore, for example, when the pressure made by the body of the driver D is applied to the polymer thick film sensor 7 on the seat belt Sb due to the body of the driver D tilting in the direction toward the steering wheel Sw, for example, the resistance value of the polymer thick film sensor 7 decreases. On the other hand, when the pressure made by the body of the driver D applied to the seat belt Sb decreases due to the body of the driver D moving away from the steering wheel Sw , the resistance value of the polymer thick film sensor 7 also increases accordingly. That is, output values from the polymer thick film sensor 7 illustrated in FIG. 7 include information on the movement direction and movement amount of the body of the driver D in the front-rear direction (Y direction) of the body.

Next, an example in which the minute vibration detection microphone 8 is attached to the seat belt Sb will be described. The minute vibration detection microphone 8 is a sensor that detects the magnitude of the sound pressure of collected sound as the magnitude of vibration. For example, when the body of the driver D is pressed against the seat belt Sb by, for example, the body of the driver D tilting in the direction toward the steering wheel Sw, the level of vibration detected by the minute vibration detection microphone 8 increases. On the other hand, when the body of the driver D moves away from the steering wheel Sw and the body of the driver D moves away from the seat belt Sb, the level of vibration detected by the minute vibration detection microphone 8 decreases. That is, output values from the minute vibration detection microphone 8 illustrated in FIG. 7 also include information on the movement direction and movement amount of the body of the driver D in the front-rear direction (Y direction) of the body.

While the embodiment and the modifications described above provide examples in which a sensor for acquiring information on the movement of the body of the driver D is attached to a component of the vehicle V, such as the seat St1 or the seat belt Sb of the vehicle V, the present invention is not limited to this. For example, an acceleration sensor may be directly attached to the body of the driver D, and the driver state detection device may be configured to compare the output value from the acceleration sensor with the output value from the acceleration sensor 1 installed in the vehicle V.

Although embodiments of the present invention have been described, the present invention is not limited to the above-described embodiments, and includes other modifications and application examples unless they deviate from the scope of the present invention described in the claims. For example, the above-described embodiments provide a detailed and specific description of the configuration of the device (driver state detection device) in order to explain the present invention in an easy-to-understand manner, and the present invention is not limited to those provided with all the configurations described above.

### REFERENCE SIGNS LIST

1 Acceleration sensor
2 Posture sensor
2a to 2d Piezoelectric sensor
3 Center-of-gravity movement amount calculation unit
4 Error signal generation unit
5 Driver state determination unit
6 Doppler sensor
7 Polymer thick film sensor
8 Minute vibration detection microphone
40x X-direction error signal generation unit
40y Y-direction error signal generation unit
41, 41x Adaptive filter
42x Delay circuit
43x Subtractor
44x Peak hold circuit
45x Average value calculation unit
51x X-direction gain adjustment unit
51y Y-direction gain adjustment unit
52 Adder
53 Threshold comparison unit
100 Driver state detection device

1. A driver state detection device comprising:
an acceleration sensor installed in a vehicle;
a center-of-gravity movement amount detection unit that is attached to a component constituting the vehicle and detects a center-of-gravity movement amount of a body of a driver on the vehicle;
a driver state determination unit that determines a state of the driver based on magnitude of an amount of deviation between an acceleration of the vehicle obtained by the acceleration sensor and the center-of-gravity movement amount of the body of the driver detected by the center-of-gravity movement amount detection unit; and
an error signal generation unit including an adaptive filter that updates a filter coefficient to reduce a value of an error signal indicating a difference between a filter output obtained by convolution of the center-of-gravity movement amount of the driver detected by the center-of-gravity movement amount detection unit and the filter coefficient and the acceleration of the vehicle obtained by the acceleration sensor, wherein
an amount of deviation between the acceleration of the vehicle and the center-of-gravity movement amount of the driver is indicated by the filter output from the adaptive filter or the value of the error signal.

2. The driver state detection device according to claim 1, wherein
the center-of-gravity movement amount detection unit comprises
a posture sensor that is provided on a seat surface of a driver's seat of the vehicle and outputs a voltage according to a pressure applied by the driver sitting on the driver's seat, and
a center-of-gravity movement amount calculation unit that calculates the center-of-gravity movement amount of the driver from an output value from the posture sensor.

3. The driver state detection device according to claim 2, wherein
the posture sensor includes four film-type piezoelectric sensors disposed in respective areas obtained by dividing a coordinate plane of the seat surface of the driver's seat into four, an X axis in the coordinate plane corresponding to a vehicle width direction of the vehicle and a Y axis in the coordinate plane corresponding to a longitudinal direction of the vehicle, and
the center-of-gravity movement amount calculation unit calculates the center-of-gravity movement amount of the driver using information on positions at which the piezoelectric sensors are arranged on the coordinate plane and output values from the piezoelectric sensors.

4. (canceled)

5. The driver state detection device according to claim 1, wherein
the center-of-gravity movement amount detection unit includes
a radio wave sensor that is provided to a seat belt to be worn by the driver and detects movement of the body of the driver by Doppler sensing.

6. The driver state detection device according to claim 1, wherein
the center-of-gravity movement amount detection unit includes
a polymer thick film sensor that is provided to a seat belt to be worn by the driver and has a characteristic of decreasing electrical resistance value with an increase in pressure applied by the body of the driver.

7. The driver state detection device according to claim 1, wherein
the center-of-gravity movement amount detection unit includes
a minute vibration detection microphone that is provided to a seat belt to be worn by the driver and detects movement of the body of the driver as magnitude of vibration.

8. The driver state detection device according to claim 1, wherein
the center-of-gravity movement amount detection unit includes
an acceleration sensor to be attached to the body of the driver.
